(19) United States
(12) Patent Application Publication
(10) Pub. No.: US 2021/0163873 A1
(43) Pub. Date: Jun. 3, 2021

(54) SYSTEM AND METHOD FOR INOCULATING AND TRANSPORTING SPECIMEN SUSPECTED TO CONTAIN NEISSERIA GONORRHOEAE OR OTHER MICROBIAL ORGANISMS

(71) Applicant: BIOMED DIAGNOSTICS, INC., White City, OR (US)

(72) Inventors: Brandon K. Font, Central Point, OR (US); John Francis Antiabong, Medford, OR (US)

(73) Assignee: BIOMED DIAGNOSTICS, INC., White City, OR (US)

(21) Appl. No.: 17/097,698

(22) Filed: Nov. 13, 2020

Related U.S. Application Data
(60) Provisional application No. 62/943,190, filed on Dec. 3, 2019.

(51) Int. Cl. C12M 1/34; C12N 1/20

(52) U.S. Cl. CPC C12M 41/34; C12N 1/20

(57) ABSTRACT

A system for inoculating and transporting Neisseria gonorrhoeae, or other microbial organisms enables inoculation and transport of a specimen suspected of containing Neisseria gonorrhoeae with an InTray® GC tray. The tray comprises a floor wall having a first depression and a second depression. A Gonococcal medium is disposed in the first depression. A removable inner panel overlays the first and second depressions. A removable outer panel overlays the inner panel. After introducing Neisseria gonorrhoeae or a microbial organism into the Gonococcal medium, the panels reseal the tray. Neisseria gonorrhoeae is introduced into the Gonococcal medium for inoculation thereof. A carbon dioxide member in the second depression releases a CO₂ gas. A carbon dioxide seal covers the second depression. The seal is punctured to release the CO₂ gas that saturates the medium in the first depression. The CO₂ gas inhibits degradation of the Gonococcal medium or other microbes that require CO₂.

### CROSS-REFERENCE TO RELATED APPLICATIONS

This application claims the benefits of U.S. provisional application No. 62/943,190, filed Dec. 3, 2020 and entitled SYSTEM AND METHOD FOR INOCULATING AND TRANSPORTING SPECIMEN SUSPECTED TO CONTAIN NEISSERIA GONORRHOEAE OR OTHER MICROBIAL ORGANISMS, which provisional application is incorporated by reference herein in its entirety.

### FIELD OF THE INVENTION

The present invention relates generally to a system and method for inoculating and transporting a specimen suspected to contain Neisseria gonorrhoeae or other microbial organisms. More so, the present invention relates to a system and method for inoculating and transporting specimen suspected to contain Neisseria gonorrhoeae or other microbial organisms through an InTray® GC tray; whereby the tray forms depressions sized and dimensioned to contain a Gonococcal medium for retaining the Neisseria gonorrhoeae, and an integrated CO₂ tablet that releases CO₂ gas, so as to inhibit degradation of the Neisseria gonorrhoeae while inoculated in the InTray® GC during study, storage, colonization, and transport operations.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Generally, Neisseria gonorrhoeae is a species of Gram-negative diplococcic bacteria responsible for causing the sexually transmitted infection gonorrhea. Diagnosis of gonorrhea is usually achieved via a laboratory test including an overnight culture. Neisseria gonorrhoeae testing and detection often involve nucleic acid amplification tests (NAAT) which, although highly sensitive and specific, have limitations regards antibiotic susceptibility testing. The benefits of NAAT, particularly its increased sensitivity when screening urine, urogenital and pharyngeal swabs, have led to its increased popularity and the subsequent decrease in bacterial culturing in clinical settings. Culturing is necessary not only to test susceptibility to antibiotics, but also to monitor treatment failures and outbreaks. Neisseria gonorrhoeae cultures, however, require specific conditions to optimize their survival, including being inoculated onto nutritive and selective chocolate agar and incubated from 35-37° in a CO₂ enriched environment.

With ceftriaxone resistance and gonorrhea incidence both on the rise, antibiotic resistance monitoring is more important than ever. Public health efforts must focus on developing new, more advanced technologies to monitor resistance of Neisseria gonorrhoeae, particularly to ceftriaxone and extended cephalosporins. Because of the difficulty of growing and recovering N. gonorrhoeae, growth media and transport systems should be evaluated for safety, efficacy and must be optimized for maximum recovery.

Prior studies have examined the difference in viability between different commercially available transport systems and generally found that the InTray GC®, a CO₂ generating packaging medium manufactured by BioMed Diagnostics, had the highest rate of recovery after extended amounts of transport times. Additionally, InTray GC plates have a shelf life of approximately one year, so are ideal for use in settings where standard chocolate agar plates may not be widely available or viable after storage.

Other proposals have involved biological culture growth and observation devices. The problem with these devices is that they do not provide an integrated carbon dioxide gas to inhibit degradation of the growth medium. Also, these biological culture growth and observation devices do not provide a stable environment for transport of the Neisseria gonorrhoeae or other microbial organisms through an InTray® GC tray. Even though the above cited biological culture growth and observation devices meet some of the needs of the market, a system and method for inoculating and transporting a specimen suspected to contain Neisseria gonorrhoeae or other microbial organisms that utilizes an InTray® GC tray; whereby the tray forms depressions sized and dimensioned to contain a Gonococcal medium for retaining the Neisseria gonorrhoeae, and an integrated CO₂ tablet that releases CO₂ gas, so as to inhibit degradation of the Neisseria gonorrhoeae while inoculated in the InTray® GC during study, storage, colonization, and transport operations, is still desired.

### SUMMARY

Illustrative embodiments of the disclosure are generally directed to a system and method for inoculating and transporting a specimen suspected to contain Neisseria gonorrhoeae or other microbial organisms. In some embodiments, the system for inoculating and transporting Neisseria gonorrhoeae comprises a tray, such as an agar plate or petri dish, used to inoculate and observe biological growth. The tray has a floor wall that defines a first depression and a second depression. The tray also has multiple sidewalls that extend up to a perimeter edge that forms an opening.

In another embodiment, the system provides a medium that is disposed in the first depression. The medium may include a Gonococcal medium. Neisseria gonorrhoeae or a microbial organism is introduced into the Gonococcal medium, so as to inoculate the Gonococcal medium.

In yet another embodiment, the system includes a carbon dioxide member disposed in the second depression. The carbon dioxide member is configured to release a carbon dioxide gas. In some embodiments, a carbon dioxide seal is configured to seal the second depression. The seal may be punctured to release a carbon dioxide gas that saturates the medium in the first depression. The carbon dioxide seal is punctured to actuate a slow release of CO₂ gas. This serves to inhibit degradation of the Gonococcal medium or other microbes that require CO₂.

The system may also include an inner panel that overlays the first and second depressions. The inner panel is configured to be removed and reapplied over the first and second depressions. The system may also include an outer panel that overlays the inner panel. The outer panel is configured to be removed and reapplied over the inner panel. The outer panel is at least partially detached from the perimeter of the tray to access the inner panel. The inner panel is at least partially detached from the perimeter of the tray to access the medium and the carbon dioxide member. After introducing Neisseria gonorrhoeae or a microbial organism into the Gonococcal medium, the panels are reattached to seal the tray.

In another aspect, the first depression is larger than the second depression.

In another aspect, the inner panel comprises a pull tab.

In another aspect, the outer panel is defined by an outer face and an adhesive face, the adhesive face engaging the inner panel.

In another aspect, the medium comprises a Gonococcal medium.

In another aspect, the Gonococcal medium is configured to grow Neisseria gonorrhoeae or a microbial organism.

In another aspect, the carbon dioxide gas helps inhibit degradation of the Neisseria gonorrhoeae or the microbial organism.

In another aspect, the system further comprises a swab operable to inoculate the Gonococcal medium with the Neisseria gonorrhoeae or the microbial organism.

In another aspect, the Gonococcal medium is defined by a chocolate color.

In another aspect, the carbon dioxide member is configured to release about 5% to 7% of the carbon dioxide gas.

In another aspect, the carbon dioxide member comprises a carbon dioxide tablet sized and dimension to form a snug fit inside the second depression.

In another aspect, the system further comprises a tool operable to puncture the carbon dioxide seal, whereby the released carbon dioxide gas engages the medium.

In another aspect, the tool comprises a sterile forceps.

In another aspect, the tray comprises an InTray Gonococcal tray.

In another aspect, the tray comprises a microbiology agar plate.

In another aspect, the tray forms a chamber that is a polypropylene cassette having a 12-month shelf life.

In another aspect, the outer panel comprises an outer adhesive label and an inner seal pull tab.

In another aspect, the GC medium is chocolate colored. Other relevant medium may be of various colors depending on their chemical composition however.

In another aspect, the GC medium or other types of microbial medium receives the specimen suspected to contain Neisseria gonorrhoeae or other relevant microbes through a 10 micro liter loop, cotton swab or synthetic fiber flocked swab.

A method for inoculating and transporting Neisseria gonorrhoeae or a microbial organism serves to create a convenient process for sexually transmitted infection clinics to transport suspected antibiotic resistant Neisseria gonorrhoeae specimens to special labs qualified to perform such testing. The method includes an initial Step of providing a tray, the tray comprising a floor wall defining a first depression and a second depression, the tray further comprising multiple sidewalls extending up to a perimeter edge forming an opening.

The method may include an initial Step of detaching at least a portion of an outer panel from the perimeter edge of the tray.

The method may further comprise a Step of detaching at least a portion of an inner panel from the perimeter edge of the tray.

A Step includes identifying a first depression in the floor wall, the first depression containing a Gonococcal medium.

In some embodiments, a Step comprises introducing Neisseria gonorrhoeae or a microbial organism into the Gonococcal medium.

A Step includes identifying a second depression in the floor wall, the second depression containing a carbon dioxide member, the second depression being sealed with a carbon dioxide seal, the carbon dioxide member being configured to release a carbon dioxide gas.

In some embodiments, a Step may include puncturing the carbon dioxide seal, whereby the carbon dioxide gas saturates the Gonococcal medium.

A Step comprises inoculating the Gonococcal medium with the Neisseria gonorrhoeae or the microbial organism.

The method may further comprise a Step of reattaching the inner panel to the perimeter edge of the tray, whereby the inner panel seals the first and second depressions.

A Step includes reattaching the outer panel to the perimeter edge of the tray, whereby the outer panel overlays the inner panel.

Another Step may include incubating the tray at about 35 degrees Celsius, for about 24 hours.

A final Step includes transporting the tray at a stable temperature from a first facility to a second facility.

In addition, the method may also be utilized with other microbial organisms that may or may not require CO₂ for growth.

One objective of the present invention is to provide a stable environment for storing, inoculating, and transporting a specimen suspected to contain Neisseria gonorrhoeae or other relevant microbes.

Another objective is to support the selective growth of Neisseria Gonorrhoeae bacteria from human specimens or other biological or environmental sources, while suppressing contaminating microflora.

Yet another objective is to provide a re-sealable container that is saturated with about 5-7% CO₂ from a CO₂ tablet.

Yet another objective is to provide a convenient way for sexually transmitted infection clinics to transport suspected antibiotic resistant Neisseria Gonorrhoeae or other relevant microbial specimens to special labs for further observation.

Yet another objective is to provide an easy to use tray for inoculating and transporting a specimen suspected to contain Neisseria gonorrhoeae, or other microbial organisms.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a top view of an exemplary system and method for inoculating and transporting a specimen suspected to contain Neisseria gonorrhoeae or other microbial organisms, in accordance with an embodiment of the present invention;

FIG. 2 illustrates a perspective view of an outer panel with label being detached from the tray to reveal a Gonococcal medium in accordance with an embodiment of the present invention;

FIG. 3 illustrates a perspective view of an inner panel being detached from the tray to reveal a carbon dioxide member, in accordance with an embodiment of the present invention;

FIG. 4 illustrates a perspective view of a punctured carbon dioxide seal releasing carbon dioxide gas, and a swab introducing Neisseria gonorrhoeae or a microbial organism into the Gonococcal medium, in accordance with an embodiment of the present invention;

FIG. 5 illustrates a perspective view of the Gonococcal medium being cross streaked with a 10 μl loop for colony isolation, in accordance with an embodiment of the present invention;

FIG. 6 illustrates a flowchart of a validation procedure of an exemplary method for inoculating and transporting Neisseria Gonorrhea or other microbial organisms in accordance with an embodiment of the present invention;

FIG. 7 illustrates a graph that references time in hours versus Log 10 CFU/mL for the InTray GC, in accordance with an embodiment of the present invention;

FIG. 8 illustrates a graph that references time in hours versus Log 10 CFU/mL for the Copan ESwab (Predicate Device), in accordance with an embodiment of the present invention; and

FIG. 9 illustrates a graph that references the results after 72 hours on the ESwab, where 2 strains passed and 3 strains failed, in accordance with an embodiment of the present invention; and

FIG. 10 illustrates a flowchart of an exemplary method for inoculating and transporting a specimen containing Neisseria gonorrhoeae or a microbial organism, in accordance with an embodiment of the present invention.

Like reference numerals refer to like parts throughout the various views of the drawings.

### DETAILED DESCRIPTION OF THE INVENTION

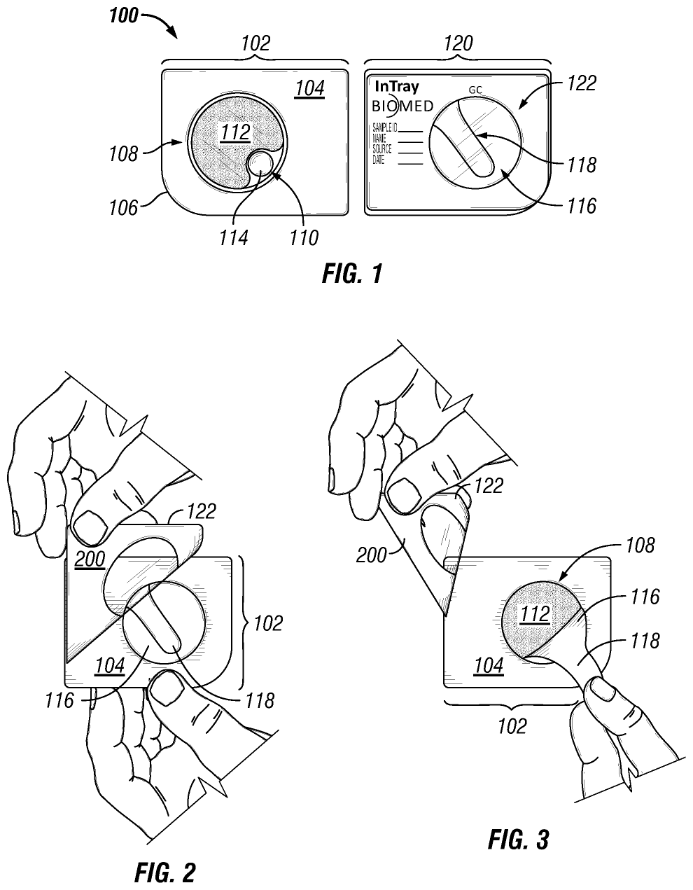

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

FIGS. 1-10 reference a system 100 and method 1000 for inoculating and transporting a specimen suspected to contain Neisseria gonorrhoeae, or other microbial organisms. The system 100 for inoculating and transporting a specimen suspected to contain Neisseria gonorrhoeae, hereafter “system 100” provides an InTray® GC (Neisseria gonorrhoeae) tray that forms depressions sized and dimensioned to contain a Gonococcal (GC) medium for supporting growth of Neisseria gonorrhoeae, and an integrated CO₂ tablet that releases CO₂ gas, so as to inhibit degradation of the Neisseria gonorrhoeae while inoculated in the InTray® GC during study, storage, colonization, and transport operations.

As referenced in FIG. 1, the system 100 comprises a tray 102 that is operational for biological functionalities, such as growing, inoculating, observing, and transporting biological elements. In one embodiment, the tray 102 is configured to retain both, a Gonococcal medium 112 for growing Neisseria gonorrhoeae or other microbial organisms, and a carbon dioxide member 114 that slowly releases a carbon dioxide (CO₂) gas to saturate the Gonococcal medium. Once the Neisseria gonorrhoeae is introduced into the medium 112, survival is optimized to enable transport inside the tray 102 in a nutritive and selective chocolate agar; and further, incubation in a temperature range between 35°-37° Celsius, and in a CO₂ enriched environment.

The tray 102 is also configured with inner and outer resealable panels 116, 122 that serve to seal the medium 112 and the carbon dioxide member 114 within the confines of the tray sidewalls 106. This unique multiple seal prevents escape of CO₂ gas; and also provides a tray 102 that is resealable, portable, and disposable. This helps facilitate inoculation and transportation of Neisseria gonorrhoeae, or other microbial organisms. In this manner, the tray 102 provides a stable environment for storing, inoculating, and transporting a specimen suspected to contain Neisseria gonorrhoeae or other relevant microbes.

In one possible embodiment, the tray 102 is defined by a generally flat, square shape. In yet another embodiment, the tray 102 is defined as a polypropylene cassette having a 12-month shelf life. In another embodiment, the tray 102 is about 2″ wide. The structure of the tray 102 includes a floor wall 104 that forms the foundation for the tray 102, and which rests on the ground surface when observing and transporting Gonococcal medium.

The floor wall 104 defines a first depression 108 and an adjacently located second depression 110. The floor wall 104 has sufficient thickness to enable formation of the depressions to a depth that is sufficient to contain a medium 112 in the first depression 108, and a carbon dioxide member 114 in the second depression 110. In one embodiment, the first depression 108 is larger than the second depression 110. The tray 102 also comprises multiple sidewalls 106 that extend up to a perimeter edge that forms an opening.

The first depression 108 that forms in the floor wall 104 may have a semi-spherical shape that is sufficiently sized, so as to support a medium 112 (See FIG. 1). In one possible embodiment, the medium 112 is efficacious for growing Neisseria gonorrhoeae or a microbial organism. In one non-limiting, the medium 112 is a Gonococcal medium. The Gonococcal medium is defined by a chocolate color. Other relevant medium may be of various colors depending on their chemical composition however.

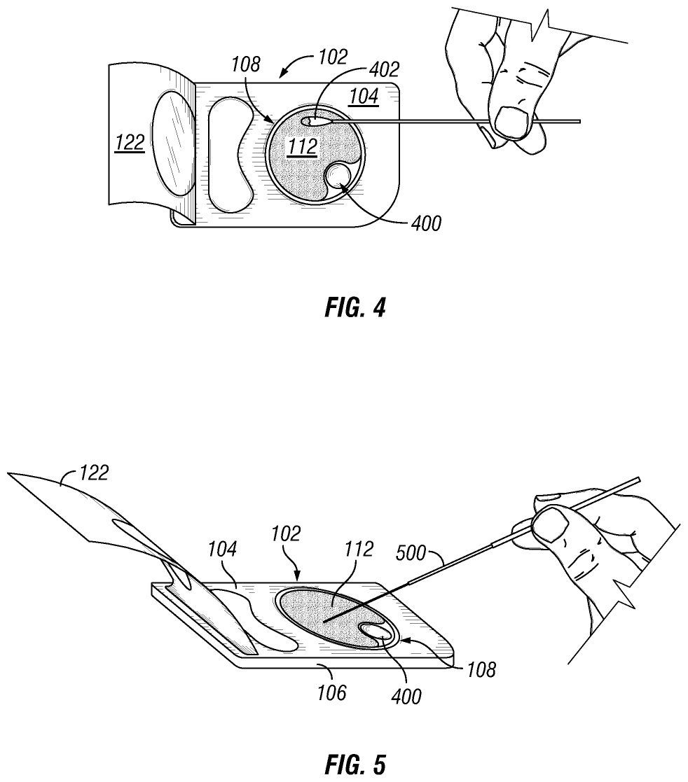

The Gonococcal medium is inoculated by introducing Neisseria gonorrhoeae or a microbial organism into the Gonococcal medium. Those skilled in the art will recognize that Neisseria gonorrhoeae is a bacterial pathogen responsible for gonorrhea and various sequelae. The introduction of Neisseria gonorrhoeae may be through use of a swab 402, or other application instrument known in the art (See FIG. 4).

In some embodiments, the swab 402 is utilized to inoculate the Gonococcal medium with the Neisseria gonorrhoeae or the microbial organism. In another embodiment, the medium 112 receives the specimen suspected to contain Neisseria gonorrhoeae or other relevant microbes through a 10 micro liter loop, cotton swab, or synthetic fiber flocked swab. For example, FIG. 5 illustrates a perspective view of the Gonococcal medium being cross streaked with a 10 μl loop for colony isolation.

In yet another embodiment, the system 100 includes a carbon dioxide member 114 disposed in the second depression 110. In some embodiments, the carbon dioxide member 114 is a carbon dioxide tablet that is sized and dimension to form a snug fit inside the second depression 110. The carbon dioxide member 114 is configured to release a carbon dioxide gas (CO₂). Those skilled in the art recognize that CO₂ gas is a stable gas. Thus, the released CO₂ gas inhibits degradation of the Neisseria gonorrhoeae in the specimen inoculated in the medium 112.

In some embodiments, a carbon dioxide seal 400 is configured to seal the second depression 110. The carbon dioxide seal 400 may be punctured to enable the slow and uniform release of CO₂ gas that saturates the medium 112 in the first depression 108. The carbon dioxide member 114 is configured to release about 5% to 7% of the CO₂ gas. This serves to inhibit degradation of the Gonococcal medium or other microbes that require CO₂. The CO₂ gas also works to inhibit degradation of the Neisseria gonorrhoeae or the microbial organism. For example, contaminating microflora is suppressed by the CO₂ gas. FIG. 4 illustrates a punctured carbon dioxide seal 400 releasing carbon dioxide gas. In one embodiment, a tool 500 is used to puncture the carbon dioxide seal 400. In some possible embodiments, the tool 500 may comprise sterile forceps.

Looking back at FIG. 2, the tray 102 also utilizes an inner panel 116 that overlays the first and second depressions 108, 110. The inner panel 116 is configured to be removed and reapplied over the first and second depressions 108, 110, so as to selectively cover the opening in the tray 102. In one possible embodiment, the inner panel 116 comprises a pull tab 118 to provide a gripping surface for the fingers when pulling one edge or corner of the inner panel 116.

The tray 102 may also include an outer panel 120 that overlays the inner panel 116. In one embodiment, the outer panel 120 is defined by an outer face 122 and an adhesive face 200. The adhesive face 200 is oriented to engage the inner panel 116, while the outer face 122 faces outwardly, so as to provide a marking surface for labeling and identifying the type of medium 112. Exemplary labels may include, without limitation, concentrations, date and time stamps, patient identification number or name, laboratory name, and clinic name.

As shown in FIG. 3, the outer panel 120 is configured to be removed and reapplied over the inner panel 116. The outer panel 120 is at least partially detached from the perimeter of the tray 102 to access the inner panel 116. The inner panel 116 is at least partially detached from the perimeter of the tray 102 to access the medium 112 and the carbon dioxide member 114. After introducing Neisseria gonorrhoeae or a microbial organism into the Gonococcal medium, the panels are reattached to seal the tray 102 for transport, and also to restrict escape of CO₂ gas.

In one exemplary experiment involving the system 100, a comparison was made between two transport systems for the recovery of viable isolates of Neisseria gonorrhoeae after varying transport times. These transport systems include: BioMed Diagnostics' InTray GC® and Copan Diagnostics' Liquid Amies Elution Swab (ESwab®) Collection and Transport System. Experimental groups underwent simulated transport conditions before being plated on standard chocolate agar, and their colony-forming units (CFU) were calculated and averaged after 0, 24, 48, and 72 hours of transport, respectively.

These averages were used to calculate loss of colonies over time, and strain viability was confirmed using inoculum verification plates for both the Intray GC and ESwab. The InTray GC plates lost fewer viable isolates of N. gonorrhoeae over 72 hours of simulated transport than the ESwab lost over only 24 hours of transport. These findings suggest that the InTray GC can be used for longer periods of transport without significant loss of viable colonies.

It is also understood that Neisseria gonorrhoeae is a gram-negative obligate human pathogen and the etiological agent of gonorrhea. Gonorrhea is not typically fatal, but if left untreated, can lead to various symptoms and significant complications, such as pelvic inflammatory disease, sterility, ectopic pregnancy, or, rarely, sepsis or death. Behind chlamydia, gonorrhea is the second most commonly reported notifiable infection in the US and experienced an 18.6% increase in incidence in 2016 alone. As of 2016, the global incidence of infection with N. gonorrhoeae was 87 million cases per year.

It is known in the art that some Neisseria gonorrhoeae strains have quickly developed resistance to many available antibiotics, often limiting the available treatment options. A combination of intramuscular ceftriaxone and oral azithromycin is generally the accepted first-line drug therapy for anogenital and pharyngeal gonorrhea in adults. In the United States, antibiotic resistance trends are monitored by the CDC's Gonococcal Isolate Surveillance Program (GISP), which has used resistance surveillance data since the 1980s to stop recommending penicillin, tetracycline, fluoroquinolones, cefixime, and other oral cephalosporins to treat gonorrhea. In recent years, strains with high resistance to ceftriaxone have arisen in several countries, severely limiting treatment options for patients infected with these particularly resistant strains.

Neisseria gonorrhoeae testing and detection often involve nucleic acid amplification tests (NAAT) which, although highly sensitive and specific, have limitations regards antibiotic susceptibility testing. The benefits of NAAT, particularly its increased sensitivity when screening urine, urogenital and pharyngeal swabs, have led to its increased popularity and the subsequent decrease in bacterial culturing in clinical settings. Culturing is necessary not only to test susceptibility to antibiotics, but also to monitor treatment failures and outbreaks. Neisseria gonorrhoeae cultures, however, require specific conditions to optimize their survival, including being inoculated onto nutritive and selective chocolate agar and incubated from 35-37° in a CO₂ enriched environment.

With ceftriaxone resistance and gonorrhea incidence both on the rise, antibiotic resistance monitoring is more important than ever. Public health efforts must focus on developing new, more advanced technologies to monitor resistance of Neisseria gonorrhoeae, particularly to ceftriaxone and extended cephalosporins. Because of the difficulty of growing and recovering N. gonorrhoeae, growth media and transport systems should be evaluated for safety, efficacy and must be optimized for maximum recovery.

Prior studies have examined the difference in viability between different commercially available transport systems and generally found that the InTray GC®, a CO₂ generating packaging medium manufactured by BioMed Diagnostics, had the highest rate of recovery after extended amounts of transport times. Additionally, InTray GC plates have a shelf life of approximately one year, so are ideal for use in settings where standard chocolate agar plates may not be widely available or viable after storage.

Thus, in an effort to build upon these previous studies, it is necessary to study how long viable clinical isolates of N. gonorrhoeae can be transported in the InTray GC tray after an initial period of incubation. To answer this question, the present system was used to compare the loss of viable cultures over 72 hours between the InTray GC and the Liquid Amies Elution Swab (ESwab) Collection and Transport System, manufactured by Copan Diagnostics.

The experimentation utilizes Neisseria gonorrhoeae AR Bank isolates #0165, #0181, #0197, and #0202 from the CDC and FDA Antibiotic Resistance Isolate bank.⁸ These three isolates are beta-lactamase negative with demonstrated susceptibility to ceftriaxone, and minimum inhibitory concentrations to azithromycin of 1, 256, and 4 μg/mL, respectively. We also used isolate ATCC 43069 from the nonprofit company American Type Culture Collection, located in Manassas, Va., USA.

The experimental groups consisted of N. gonorrhoeae inoculated onto the InTray GC transport devices (BioMed Diagnostics, Inc., OR, USA), and into the Liquid Amies Elution Swab (ESwab) Collection and Transport System (Copan Diagnostics, CA, USA). Inoculum verification was also performed on standard chocolate agar plates to determine the viability of the experimental strains. Furthermore, a null test was performed to observe colony loss on the ESwab after 72 hours, although the manufacturer only claims specimen viability for up to 24 hours.

Colony-forming units (CFUs) were calculated using spectrophotometers at 625 nm adjusted to a 0.5 McFarland standard in 0.85% physiological saline, as per CSLI guidelines.⁹ We calculated the mean log 10 CFU/mL for each time point by taking the average of both duplicates of each dilution, then averaging all the dilutions within each triplicate. From there, we averaged the log 10 CFU/mL of all three triplicates (A, B, and C) to create a single value for the time point.

A percent decrease was calculated for each strain in both experimental groups by subtracting the final log 10 CFU/mL value from the initial log 10 CFU/mL value, dividing this value by the initial log 10 CFU/mL quantity, then multiplying by 100%. The percent decreases were then compared between the InTray GC and the ESwab to see how many viable colonies were lost over time. Strain failure was determined by a 100% decrease in viable colonies after 72 hours in either experimental group.

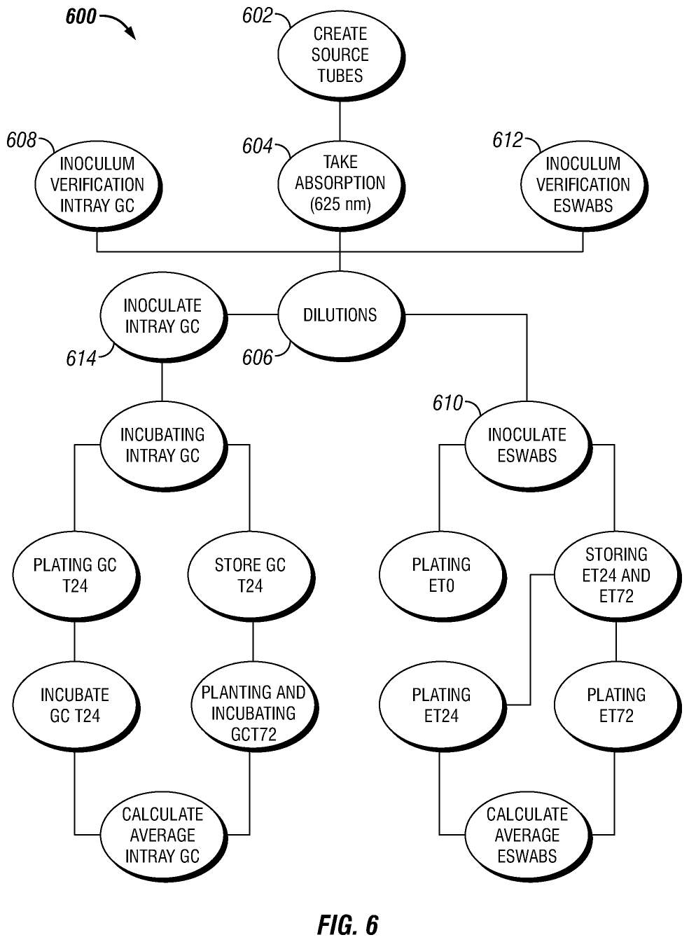

FIG. 6 illustrates a flowchart diagram of an exemplary method 600 for inoculating and transporting Neisseria gonorrhea. The method 600 initiates with a Step 602 of creating source tubes for each respective isolate (AR 0165, ATCC 43069, AR 0181, AR 0197, AR 0202) by separately inoculating standard chocolate agar plates with each isolate, then incubating each at 37° C. in a 5-7% CO₂ environment for 24 hours. The colonies are then picked from individual plates with a 1 μL loop and mixed each into separate vials of 1.2 mL of a 0.85% saline solution, before a Step 604 of measuring the mean average absorption with a blanked spectrophotometer at 625 nm adjusted to a 0.5 McFarland standard. This process was completed twice for each isolate to create two source tubes, then a Step 606 include making serial dilutions from each to achieve concentrations of 10⁷, 10⁶, 10⁵, 10⁴, 10³, 10^{2.69}, 10^{2.39}, and 10² CFU/mL.

For each individual isolate, 9 InTray GC plates (triplicates from 3 lots; A, B, and C) are inoculated with 20 μL from the 10³ dilution according to manufacturer instructions, before placing them into a 37° C. incubator. 24 hours later, we removed the plates, counted and recorded the colonies on each, then chose the six most homogenous plates (2 from each lot A, B, and C). The two most similar counts from each lot were picked for downstream analysis and the higher count was tagged as GC t24 (time 24 hours) to avoid over-estimation viability bias for GC t72.

The 3 GC t24 plates are then plated by scraping and removing all colonies from each plate (starting with A) with a 1 μL loop and swirling into 1 mL of 0.85% saline, followed by serial dilutions in the range of 10⁴ to 10⁰ CFU/mL. After, we plated 100 μL from each dilution in duplicates onto standard chocolate agar plates and placed them into the CO₂ enriched incubator.

At the same time, three GC t72 plates were placed into a therapak-biohazard-specimen transport bag and the bag was placed into the trunk of a car with a USB temperature-data logger to simulate transport. Two days later, the plates are removed from the trunk and repeated the plating and dilution process that were used for GC t24, and then placed these plates into the CO₂ enriched incubator. Two days after incubating GC t24 and one day after incubating GC t72, we removed them from the incubators and recorded the plate counts in the range of 30-250 CFUs.

In addition to the method with InTray GC 608, the method is also employed to test the viability of Copan ESwabs' 612 over time. For each isolate, we began by inoculating 3 sets of triplicate ESwabs (Et₀ A, B, C; Et₂₄ A, B, C; Et₇₂ A, B, C) by placing each swab into a separate test tube with 100 μL of the 10⁷ CFU/mL dilution for 10-15 seconds, before replacing each swab into their respective container of Liquid Amies. The triplicates of Et₂₄ and Et₇₂ were then placed in the trunk of a car, under similar conditions to GC t72.

Triplicates of Et₀ were given about 5-15 minutes to rest before each was vortexed and the 100 μL of liquid from each swab into separate 5 mL test tubes is expressed. Then, serial dilutions (10⁴, 10³, 10²⁶⁹, 10²³⁹, and 10² CFU/mL) were created for each set (A, B, and C), plated 100 μL onto standard chocolate agar plates in duplicates, and placed these into the CO₂ incubator. 24 hours later, the plates were removed from the incubator and the CFU per plate was recorded. The same day, we removed the Et₂₄ plates from the trunk and repeated the plating, dilution, and incubation process from Et₀, and repeated this process again for the Et₇₂ plates 72 hours after placing them in the trunk.

In order to demonstrate strain viability, inoculum verification 610 was performed for the InTray GC and ESwab. For the InTray GC, 100 μL was spread from the 10⁴, 10³, 10^{2.69}, 10^{2.39}, and 10² CFU/mL dilutions onto duplicate standard chocolate agar plates (10 plates total) and incubated them in the CO₂ enriched incubator for 24 hours. After 24 hours, each inoculum verification plates was removed and recorded 614 with the duplicate's plate count in the range of 30-250 CFU per plate. This process was repeated for the ESwab inoculum verification, but returned the IV plates to the incubator after recording the 24-hour plate counts and kept them there for an additional 24 hours before counting them again, in order to form a null comparison test for Et₇₂.

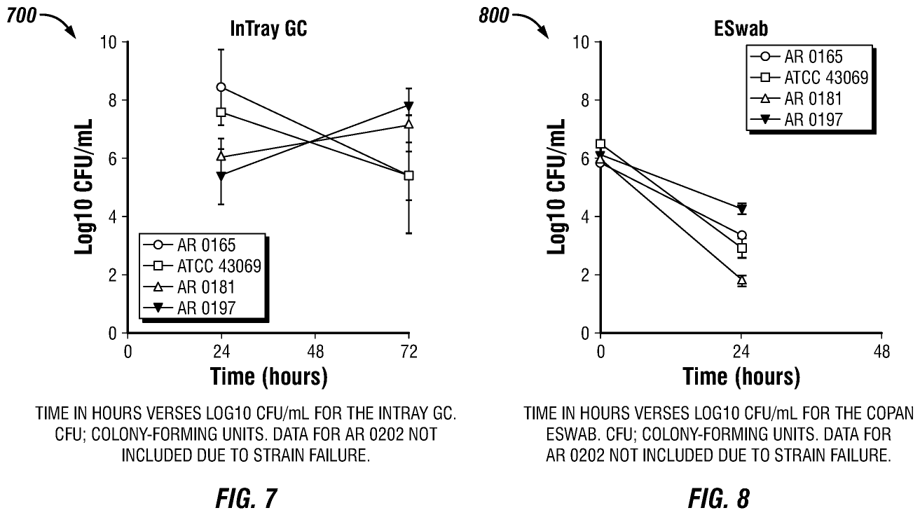

FIGS. 7-9 are graphs that illustrate the results of the aforementioned experiments in the method 600. The charts show an X-axis for time in hours; and a Y-axis that shows CFU/mL at a Log 10. In a result for the InTray GC, shown in FIG. 7, the 5 strains are tested on the InTray GC at 72 hours. The illustrated result shows that 4 strains passed and 1 strain failed (AR 0202). AR 0165 experienced a 16.28% decrease in log 10 CFU/mL from 24 to 72 hours; ATCC 43069 had a slightly smaller percent decrease of 12.33%. The other two strains, AR 0181 and AR 0197, actually had increases in their log 10 CFU/mL over the same time period, with percent increases of 6.78% and 15.50%, respectively. When averaged, the percent decrease across successful strains after 72 hours on the InTray GC, was 1.58%. Graph 700 references, time in hours versus Log 10 CFU/mL for the InTray GC. CFU; colony forming units. Data for AR 0202 not included because of strain failure.

In a result for ESwab, shown in FIG. 8, the 5 strains are tested on the ESwab, after 24 hours, 4 strains passed and 1 strain failed (AR 0202). After 24 hours on the ESwab, AR 0165 decreased in log 10 CFU/ml by 15.9%, ATCC 43069 decreased by 21.62%, AR 0181 decreased by 26.27%, and AR 0197 decreased by 11.25%. When averaged, the percent decrease across successful strains after 24 hours on the ESwab, was 18.76%. Graph 800 references, time in hours versus Log 10 CFU/mL for the Copan ESwab. CFU; colony-forming units. Data for AR 0202 not included because of strain failure.

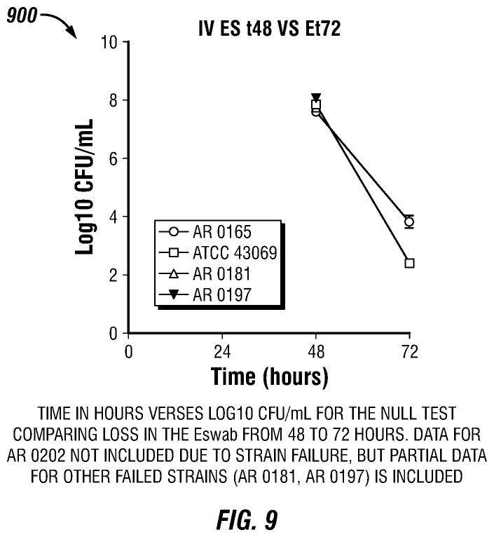

Turning now to FIG. 9, graph 900 shows the results after 72 hours on the ESwab, where 2 strains passed and 3 strains failed (AR 0181, AR 0197, AR 0202). Of the two passing strains, AR 0165 experienced a 49.87% decrease in viable colonies from 48 to 72 hours. ATCC 43069 experienced a larger percent decrease of 69.41% over the same time period. The average percent decrease between the passing strains was 59.64%. Data from all inoculum verification plates, besides successful strains on the IV plates for ESwab after 48 hours, is not shown.

Graph 900 illustrates that time in hours versus Log 10 CFU/mL for the null test comparing loss in the ESwab from 48 to 72 hours. Data for AR 0202 not included because of strain failure, but partial data for other failed strains (AR 0181, AR 0197) is included. Nonetheless, all inoculum verification tests confirmed strain viability of the experimental groups, including for strain AR 0202, which failed for all experimental groups and was therefore excluded from the figures. time in hours versus Log 10 CFU/mL for the null test comparing loss in the ESwab from 48 to 72 hours. Data for AR 0202 not included because of strain failure, but partial data for other failed strains (AR 0181, AR 0197) is included.

Thus, after 72 hours of simulated transport on the InTray GC transport systems, there was a smaller percent decrease in viable colonies when compared to only 24 hours of transport using the Copan ESwab. The minimal decrease in bacterial quantities show the InTray GC's capability to maintain a transport environment that facilitates the survival of various strains of N. gonorrhoeae.

After 72 hours held in ambient transport conditions, the InTray GC plates experienced an average 1.58% decrease in colonies, while the ESwab lost between 18.76% of colonies after 24 hours in the same conditions. The ESwab also experienced 3 strain failures after 72 hours, and the 2 strain successes had an average 59.64% decrease in viable colonies. Our experiments showed that, under controlled room temperatures, the InTray GC had better species recovery after 72 hours when compared to the ESwab after 24 hours.

This data supports a previous study examining four commercial transport systems and comparing recovery rates between the systems, which found that the InTray GC had optimal recovery of specimen in comparison to other systems. Our study builds upon this research by demonstrating that the InTray GC plates can be subjected to extended transport times up to 72 hours without significant loss of specimen viability.

This research is integral to future research on antibiotic resistance in Neisseria gonorrhoeae. The ability of the InTray GC to recover higher quantities of the bacteria after long periods of transport should ideally lead to its increased used as a transport system. With an average of 1.58% of colonies lost after 72 hours, the InTray GC could be used to transport longer distances to facilities with advanced antibiotic susceptibility testing capabilities. Considering the difficulty in maintaining viable N. gonorrhoeae cultures in transport conditions, as well as the rising rates of antibiotic resistance, it is important to recognize the InTray GC as a viable option for a transport system with high recovery rates after extended periods of time.

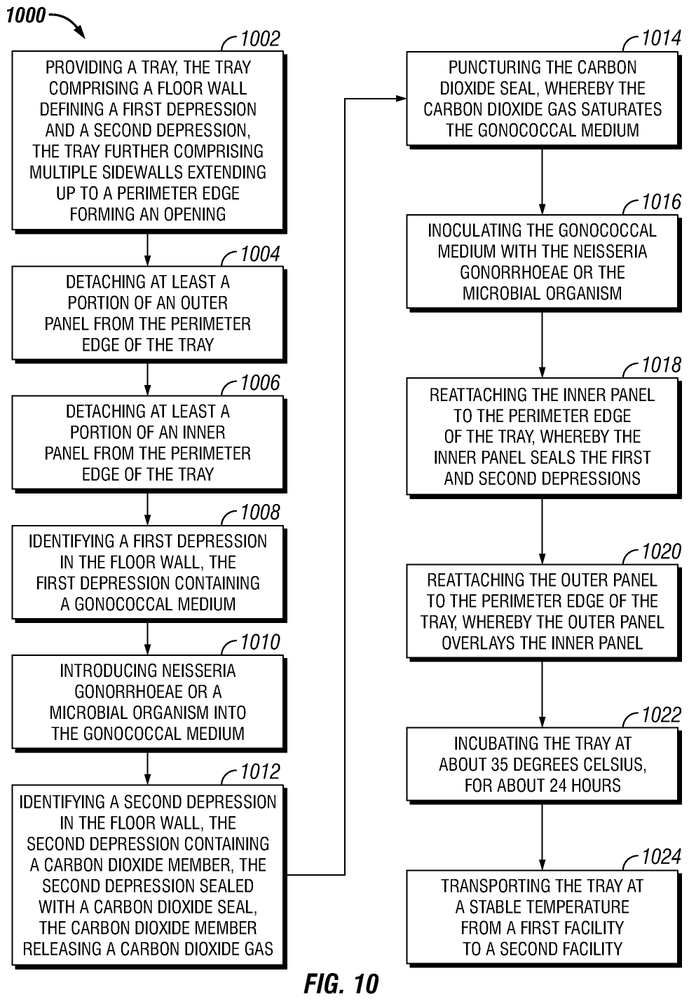

FIG. 10 illustrates a flowchart of an exemplary method 1000 for inoculating and transporting a specimen containing Neisseria gonorrhoeae or a microbial organism. In addition, the method 1000 may also be utilized with other microbial organisms that may or may not require CO₂ for growth. The method 1000 for inoculating and transporting Neisseria gonorrhoeae or a microbial organism serves to create a convenient process for sexually transmitted infection clinics to transport suspected antibiotic resistant Neisseria gonorrhoeae specimens to special labs qualified to perform such testing.

In one possible embodiment, the method 1000 comprises an initial Step 1002 of providing a tray, the tray comprising a floor wall defining a first depression and a second depression, the tray further comprising multiple sidewalls extending up to a perimeter edge forming an opening. tray 102 that is operational for biological functionalities, such as growing, inoculating, and observing biological elements in an agar plate, a petri dish, and the like. In one embodiment, the tray 102 is configured to support both, a Gonococcal medium, and a carbon dioxide member 114 that slowly releases a carbon dioxide gas to saturate the Gonococcal medium.

The method 1000 may include another Step 1004 of detaching at least a portion of an outer panel from the perimeter edge of the tray. The outer panel is resilient, and provides an outer marking surface for labeling. The method 1000 may further comprise a Step 1006 of detaching at least a portion of an inner panel from the perimeter edge of the tray. Use of the inner panel creates a dual seal security feature. A Step 1008 includes identifying a first depression in the floor wall, the first depression containing a Gonococcal medium. In some embodiments, a Step 1010 comprises introducing Neisseria gonorrhoeae or a microbial organism into the Gonococcal medium. This can be performed through a swab or other known medical or laboratory instrument.

A Step 1012 includes identifying a second depression in the floor wall, the second depression containing a carbon dioxide member, the second depression being sealed with a carbon dioxide seal, the carbon dioxide member being configured to release a carbon dioxide gas. The second depression is smaller than the first depression because the carbon dioxide member is a tablet. In some embodiments, a Step 1014 may include puncturing the carbon dioxide seal, whereby the carbon dioxide gas saturates the Gonococcal medium. The seal may be punctured to release the CO₂ gas that saturates the medium 112 in the first depression 108. For example, FIG. 4 illustrates a perspective view of a punctured carbon dioxide seal releasing carbon dioxide gas.

A Step 1016 comprises inoculating the Gonococcal medium with the Neisseria gonorrhoeae or the microbial organism. FIG. 4 also shows the medium prepared for the inoculation through swabbing. The method 1000 may further comprise a Step 1018 of reattaching the inner panel to the perimeter edge of the tray, whereby the inner panel seals the first and second depressions. A Step 1020 includes reattaching the outer panel to the perimeter edge of the tray, whereby the outer panel overlays the inner panel. This serves to seal the medium in preparation for incubation. The seal also prevents escape of the CO₂ gas, which helps prevent degradation of the medium.

Another Step 1022 may include incubating the tray at about 35° Celsius, for about 24 hours. These conditions are necessary because Neisseria gonorrhoeae cultures require specific conditions to optimize their survival, including being inoculated onto nutritive and selective chocolate agar and incubated from 35°-37° Celsius in a CO₂ enriched environment. A final Step 1024 includes transporting the tray at a stable temperature from a first facility to a second facility. In one non-limiting embodiment, this Step 1024 enables sexually transmitted infection clinics to transport suspected antibiotic resistant Neisseria gonorrhoeae specimens to special labs qualified to perform such testing.

In conclusion, system for inoculating and transporting Neisseria gonorrhoeae, or other microbial organisms enables inoculation and transport of a specimen suspected of containing Neisseria gonorrhoeae with an InTray® GC tray. The tray comprises a floor wall having a first depression and a second depression. A Gonococcal medium is disposed in the first depression. A removable inner panel overlays the first and second depressions. A removable outer panel overlays the inner panel. After introducing Neisseria gonorrhoeae or a microbial organism into the Gonococcal medium, the panels reseal the tray. Neisseria gonorrhoeae is introduced into the Gonococcal medium for inoculation thereof. A carbon dioxide member in the second depression releases a CO₂ gas. A carbon dioxide seal covers the second depression. The seal is punctured to release the CO₂ gas that saturates the medium in the first depression. The CO₂ gas inhibits degradation of the Gonococcal medium or other microbes that require CO₂.

These and other advantages of the invention will be further understood and appreciated by those skilled in the art by reference to the following written specification, claims and appended drawings.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A system for inoculating and transporting Neisseria gonorrhoeae or a microbial organism, the system comprising:
a tray comprising a floor wall defining a first depression and a second depression, the tray further comprising multiple sidewalls extending up to a perimeter edge forming an opening;
a medium disposed in the first depression;
a carbon dioxide member disposed in the second depression, the carbon dioxide member configured to release a carbon dioxide gas;
a carbon dioxide seal covering the second depression;
an inner panel overlaying the first and second depressions, the inner panel configured to be removed and reapplied over the first and second depressions; and
an outer panel overlaying the inner panel, the outer panel configured to be removed and reapplied over the inner panel.

2. The system of claim 1, wherein the first depression is larger than the second depression.

3. The system of claim 1, wherein the inner panel comprises a pull tab.

4. The system of claim 1, wherein the outer panel is defined by an outer face and an adhesive face, the adhesive face engaging the inner panel.

5. The system of claim 1, wherein the medium comprises a Gonococcal medium.

6. The system of claim 5, wherein the Gonococcal medium is configured to grow Neisseria gonorrhoeae or a microbial organism.

7. The system of claim 6, wherein the carbon dioxide gas inhibits degradation of the Neisseria gonorrhoeae or the microbial organism.

8. The system of claim 6, further comprising a swab operable to inoculate the Gonococcal medium with the Neisseria gonorrhoeae or the microbial organism.

9. The system of claim 6, wherein the Gonococcal medium is defined by a chocolate color.

10. The system of claim 1, wherein the carbon dioxide member is configured to release about 5 percent to 7 percent of the carbon dioxide gas.

11. The system of claim 1, wherein the carbon dioxide member comprises a carbon dioxide tablet sized and dimension to form a snug fit inside the second depression.

12. The system of claim 1, further comprising a tool operable to puncture the carbon dioxide seal, whereby the released carbon dioxide gas engages the medium.

13. The system of claim 1, wherein the tool comprises a sterile forceps.

14. The system of claim 1, wherein the tray comprises a polypropylene cassette having a 12-month shelf life.

15. The system of claim 1, wherein the tray comprises an InTray Gonococcal tray.

16. A system for inoculating and transporting Neisseria gonorrhoeae or a microbial organism, the system comprising:
a tray comprising a floor wall defining a first depression and a second depression, the tray further comprising multiple sidewalls extending up to a perimeter edge forming an opening;
a Gonococcal medium disposed in the first depression, the Gonococcal medium being configured to grow Neisseria gonorrhoeae or a microbial organism;
a carbon dioxide member disposed in the second depression, the carbon dioxide member configured to release a carbon dioxide gas to the Gonococcal medium;
a carbon dioxide seal covering the second depression;
a tool operable to puncture the carbon dioxide seal, whereby the released carbon dioxide gas engages the Gonococcal medium;
an inner panel overlaying the first and second depressions, the inner panel comprising a pull tab, the inner panel configured to be removed and reapplied over the first and second depressions;
an outer panel overlaying the inner panel, the outer panel being defined by an outer face and an adhesive face, the adhesive face engaging the inner panel, the outer panel configured to be removed and reapplied over the inner panel; and
a swab operable to inoculate the Gonococcal medium with the Neisseria gonorrhoeae or the microbial organism.

17. The system of claim 16, wherein the carbon dioxide gas helps inhibit degradation of the Neisseria gonorrhoeae or the microbial organism.

18. A method for inoculating and transporting a specimen containing Neisseria gonorrhoeae or a microbial organism, the method comprising:
providing a tray, the tray comprising a floor wall defining a first depression and a second depression, the tray further comprising multiple sidewalls extending up to a perimeter edge forming an opening;
detaching at least a portion of an outer panel from the perimeter edge of the tray;
detaching at least a portion of an inner panel from the perimeter edge of the tray;
identifying a first depression in the floor wall, the first depression containing a Gonococcal medium;
introducing Neisseria gonorrhoeae or a microbial organism into the Gonococcal medium;
identifying a second depression in the floor wall, the second depression containing a carbon dioxide member, the second depression being sealed with a carbon dioxide seal, the carbon dioxide member being configured to release a carbon dioxide gas;
puncturing the carbon dioxide seal, whereby the carbon dioxide gas saturates the Gonococcal medium;
inoculating the Gonococcal medium with the Neisseria gonorrhoeae or the microbial organism;
reattaching the inner panel to the perimeter edge of the tray, whereby the inner panel seals the first and second depressions;
reattaching the outer panel to the perimeter edge of the tray, whereby the outer panel overlays the inner panel; and
incubating the tray at about 35 degrees Celsius, for about 24 hours.

19. The method of claim 18 further comprising, transporting the tray at a stable temperature from a first facility to a second facility.

20. The system of claim 18, wherein the carbon dioxide member is configured to release about 5 percent to 7 percent of the carbon dioxide gas.
